(19) United States
(12) Patent Application Publication
(10) Pub. No.: US 2017/0181887 A1
(43) Pub. Date: Jun. 29, 2017

(54) MIGRAINE HAT

(71) Applicant: ALLSTAR MARKETING GROUP, LLP, HAWTHORNE, NY (US)

(72) Inventor: Thomas GENTILE, Mahopac, NY (US)

(73) Assignee: ALLSTAR MARKETING GROUP, LLP, HAWTHORNE, NY (US)

(21) Appl. No.: 15/422,710

(22) Filed: Feb. 2, 2017

Related U.S. Application Data
(60) Provisional application No. 62/321,920, filed on Apr. 13, 2016.
(60) Provisional application No. 62/262,096, filed on Dec. 2, 2015.

(30) Foreign Application Priority Data
Apr. 19, 2016 (CN) 201620330067.9

(51) Int. Cl. A61F 7/02; A42B 1/00

(52) U.S. Cl. CPC A61F 7/02; A42B 1/006; A42B 1/008; A61F 2007/0008

(57) ABSTRACT

A pain management system wearable on a head of a person may include a thermal device including a plurality of compartments, each compartment containing a material that is configured to maintain a temperature that is greater than or less than an ambient temperature for a duration of time that is greater than materials forming a remainder of the device, the compartments being generally aligned in rows and columns and including fold lines therebetween such that the thermal device is configured to approximate a curvature of the head; and a generally elongated pouch including a pocket configured to receive the thermal device therein, the generally elongated pouch configured to approximate the curvature of the head when worn and with the thermal device contained therein. A kit may include the system and may include one or more of the constituent elements of the system.

### CROSS REFERENCE

This application relates to and claims priority from U.S. Ser. No. 62/321,920 filed Apr. 13, 2016, the entire contents of which are incorporated by reference. This application also relates to and claims priority from U.S. Ser. No. 62/262,096 filed Dec. 2, 2015, the entire contents of which are incorporated herein by reference.

This application additionally relates to and claims priority from Chinese Utility Model Ser. No. 201620330067.9 filed Apr. 19, 2016, the entire contents of which are incorporated herein by reference, which in turn claims priority to U.S. Ser. No. 62/321,920 filed Apr. 13, 2016 and also claims priority from U.S. Ser. No. 62/262,096 filed Dec. 2, 2015, the entire contents of which are incorporated herein by reference.

### BACKGROUND

Technical Field

The present disclosure generally relates to a device for treating headaches and more particularly to headgear for applying thermal compresses to a head for reducing pain resulting from headaches such as migraines.

Description of the Related Art

The causes of headaches are not clearly understood. While the brain itself cannot feel pain, headaches are believed to be caused by surrounding tissues, brain chemicals, blood vessels, and nerves that produce pain signals.

One known treatment for headaches is temperature therapy in which hot or cold compresses are applied to the head or neck. Ice packs having a numbing effect, which may dull the sensation of pain. Hot packs and heating pads can relax tense muscles.

There is a continuing need for new devices and methods for treating or alleviating the symptoms associated with headaches.

### SUMMARY

A pain management system wearable on a head of a person may include a thermal device including a plurality of compartments, each compartment containing a material that is configured to maintain a temperature that is greater than or less than an ambient temperature for a duration of time that is greater than materials forming a remainder of the device, the compartments being generally aligned in rows and columns and including fold lines therebetween such that the thermal device is configured to approximate a curvature of the head; and a generally elongated pouch including a pocket configured to receive the thermal device therein, the generally elongated pouch configured to approximate the curvature of the head when worn and with the thermal device contained therein. A kit may include the system and may include one or more of the constituent elements of the system.

The above and other aspects, features and advantages of the present invention will become apparent from the following description read in conjunction with the accompanying drawings, in which like reference numerals designate the same elements.

### BRIEF DESCRIPTION OF THE DRAWINGS

A further understanding of the present invention can be obtained by reference to a preferred embodiment set forth in the illustrations of the accompanying drawings. Although the illustrated preferred embodiment is merely exemplary of methods, structures and compositions for carrying out the present invention, both the organization and method of the invention, in general, together with further objectives and advantages thereof, may be more easily understood by reference to the drawings and the following description. The drawings are not intended to limit the scope of this invention, which is set forth with particularity in the claims as appended or as subsequently amended, but merely to clarify and exemplify the invention.

For a more complete understanding of the present invention, reference is now made to the following drawings in which:

FIG. 1 is a front view of a thermal device in accordance with the present disclosure;

FIG. 2 is a perspective view of an elongated pouch in accordance with the present disclosure;

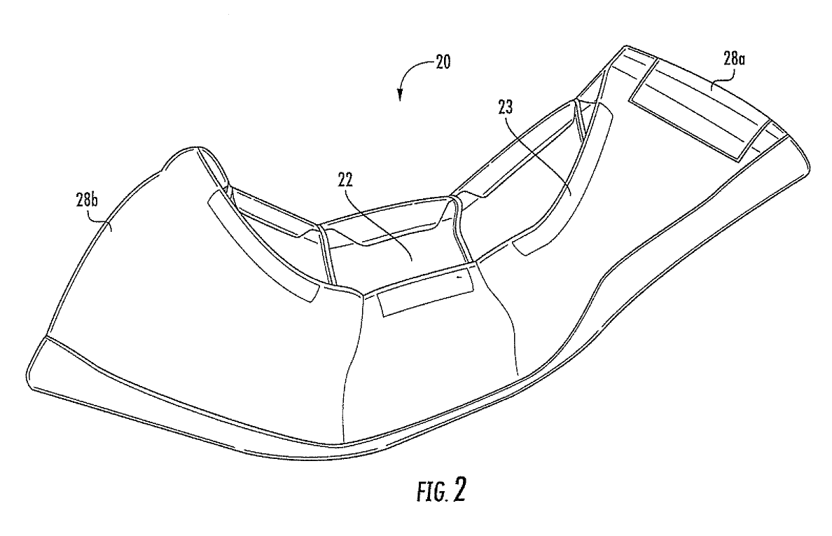

FIG. 3 is a perspective view of the thermal device, elongated pouch, and a cap being worn by a user; and

FIG. 4 is a perspective view of a kit containing the portions of the migraine hat system in accordance with the present disclosure;

FIG. 5 is a pouch for containing the portions of the migraine hat system;

FIG. 6 is a rear view of the pouch of FIG. 5;

FIG. 7 is an open view of the pouch of FIG. 5; and

FIG. 8 is a further top view of the pouch of FIG. 5, with the flap open but the inner closure shown covering the opening.

### DETAILED DESCRIPTION OF THE PREFERRED EMBODIMENTS

As required, a detailed illustrative embodiment of the present invention is disclosed herein. However, techniques, systems, compositions and operating structures in accordance with the present invention may be embodied in a wide variety of sizes, shapes, forms and modes, some of which may be quite different from those in the disclosed embodiment. Consequently, the specific structural and functional details disclosed herein are merely representative, yet in that regard, they are deemed to afford the best embodiment for purposes of disclosure and to provide a basis for the claims herein which define the scope of the present invention.

Reference will now be made in detail to several embodiments of the invention that are illustrated in the accompanying drawings. Wherever possible, same or similar reference numerals are used in the drawings and the description to refer to the same or like parts or steps. The drawings are in simplified form and are not to precise scale. For purposes of convenience and clarity only, directional terms, such as top, bottom, up, down, over, above, below, etc., or motional terms, such as forward, back, sideways, transverse, etc. may be used with respect to the drawings. These and similar directional terms should not be construed to limit the scope of the invention in any manner.

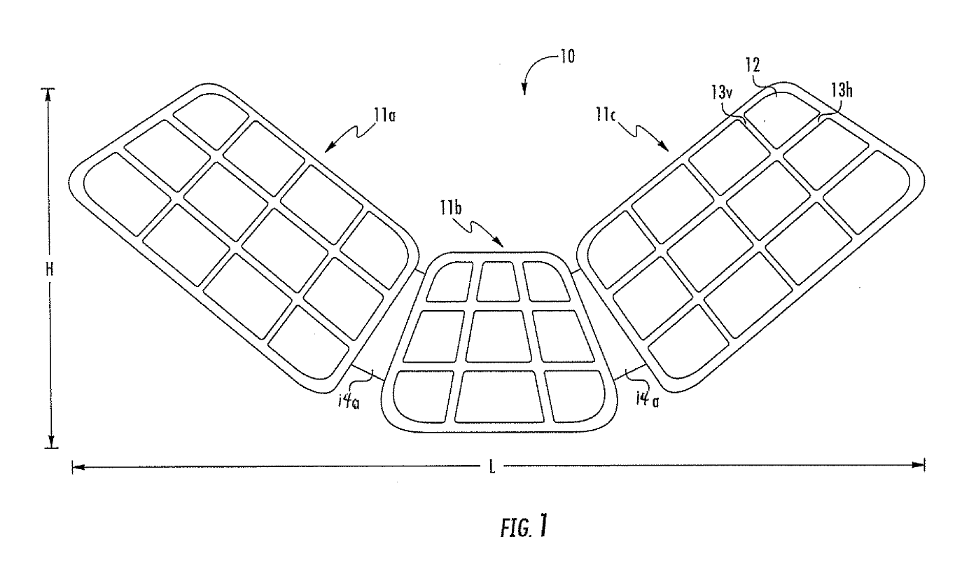

Referring to FIG. 1, a thermal device 10 will now be described. The thermal device 10 may include a plurality of sections including, for example, a first section 11a, a second section 11b, and a third section 11c. The device 10 has an overall length L which may be, for example, 22 inches and an overall height H which may be, for example, 11 inches. The first section 11a and third section 11c may be shaped and sized to be worn on the sides of a user's head. The second section 11b be shaped and sized to be worn at the back of the user's head. The first section 11a may be connected to the second section 11b by a first piece of bridging material 14a, which may be stretchable and/or elastic. The second section 11b may be connected to a third section 11c by a second piece of material 14b, which is substantially similar to the first piece of material 14a.

Each of the sections 11a-c may include a plurality of compartments 12 that are generally arranged in rows and columns such that fold lines 13h, 13v are disposed between adjacent one of the compartments 12 such that the sections 11a-c may bend to conform to a curvature of a user's head. Each of the compartments 12 may include a material or substance that may be cooled or heated such as a gel material that may be frozen, cooled, and/or heated and maintains its temperature for a relatively long duration of time as compared to the remainder of the device. For example, the compartments 12 may contain a refrigerant gel or liquid such as water, ammonium nitrate, diethylene glycol, and/or ethelene glycol. Nontoxic substances may be preferably used.

The thermal device 10 is configured to be releasably received within a pouch 20 which may be formed from a fabric material. For example, an outer surface of the thermal device 10 may be formed from a hook and loop type fastener connection or VELCRO compatible neoprene material or the like, and an inner surface of the thermal device 10 (which would come in contact with a user's body) may be formed from a cotton and/or spandex blend. The pouch 20 may include a pocket 22 in which the thermal device 10 may be received and one or more surfaces including VELCRO may be positioned at the open end of the pocket 22 to releasably seal and contain the thermal device 10 within the pocket 22. A flap 24a may be releasably secured at a designated area 24b of the device 20 to secure the pouch 20 with the thermal device 10 contained therein to the head of a user.

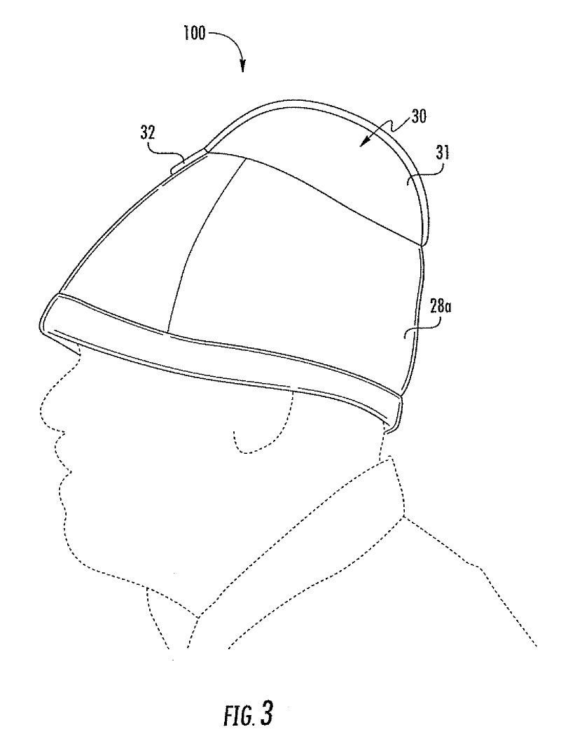

As shown in FIG. 3, a user P is shown wearing a pain management system 100 including pouch 20 containing the thermal device 10 and an end cap 30. With the pouch 20 containing the thermal device 10, the pouch 20 may be secured to a head of user P by adjusting the flap 24a to snuggly secure the pouch 20 to the head. A cap 30 including a conical section 32 and VELCRO flap(s) 32 may be releasably secured to a top end or portion of the pouch 20. The cap 30 may be insulated and/or may include a compartment such as compartments 12 which may contain a substance that may be heated and/or cooled.

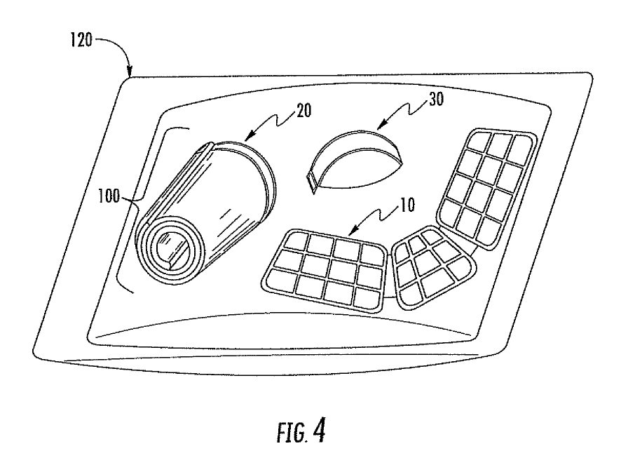

As shown in FIG. 4, a kit 120 may include the system 100. The kit 120 may include the system 100 and may include one or more of the constituent elements of the system. The kit 120 may include a plurality or variety of thermal devices 10 such that as one of the thermal devices 10 becomes too warm or cool, another of the devices 10 may be placed within the pouch 20 for use. Alternatively, some of the devices 10 may be better suited for being heated and other of the devices 10 may be better suited for being cooled. Thus, the user P may select an appropriate device 10 from the kit 120 as desired.

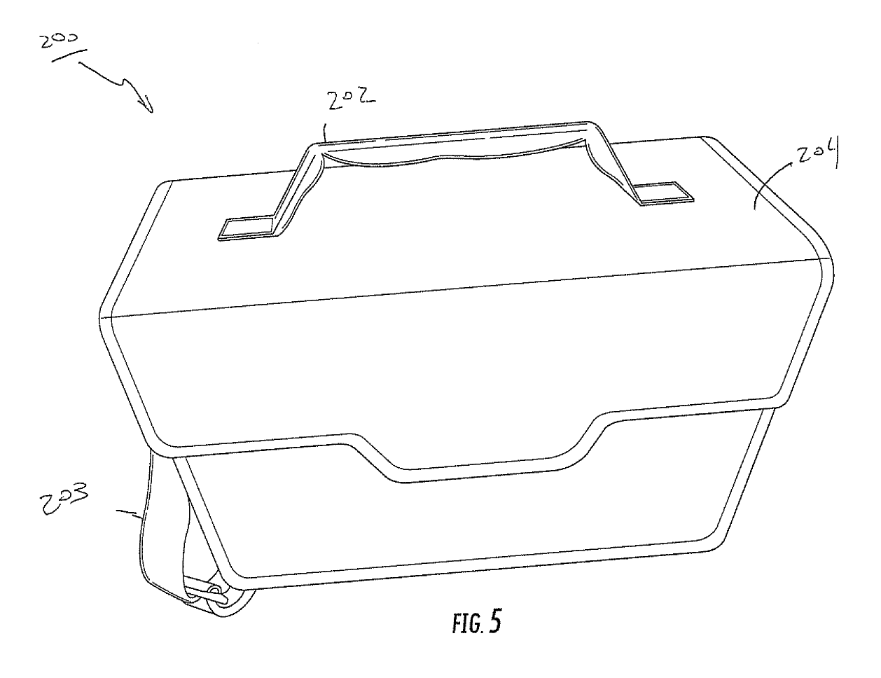

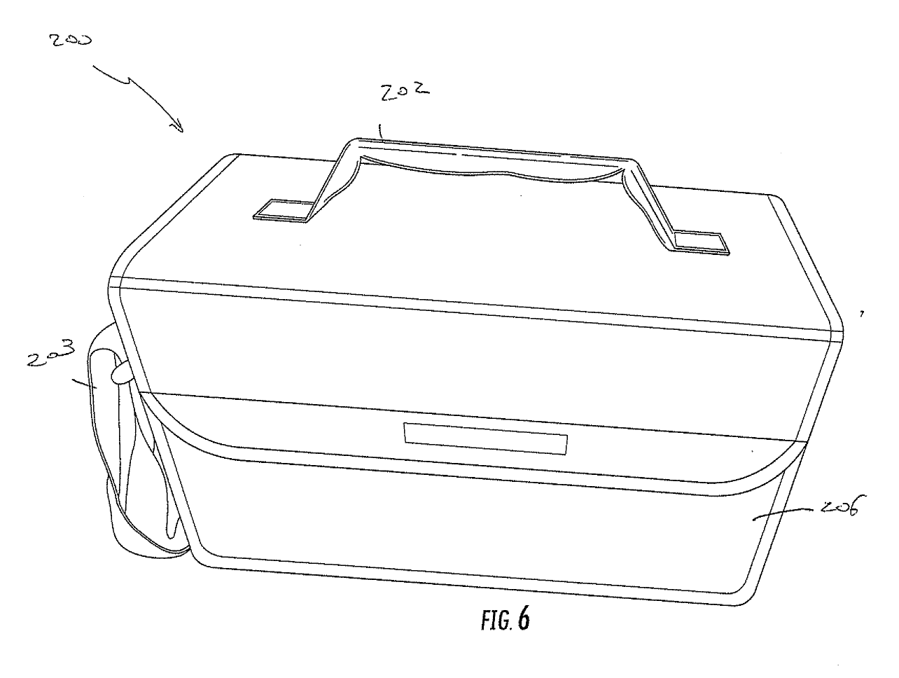

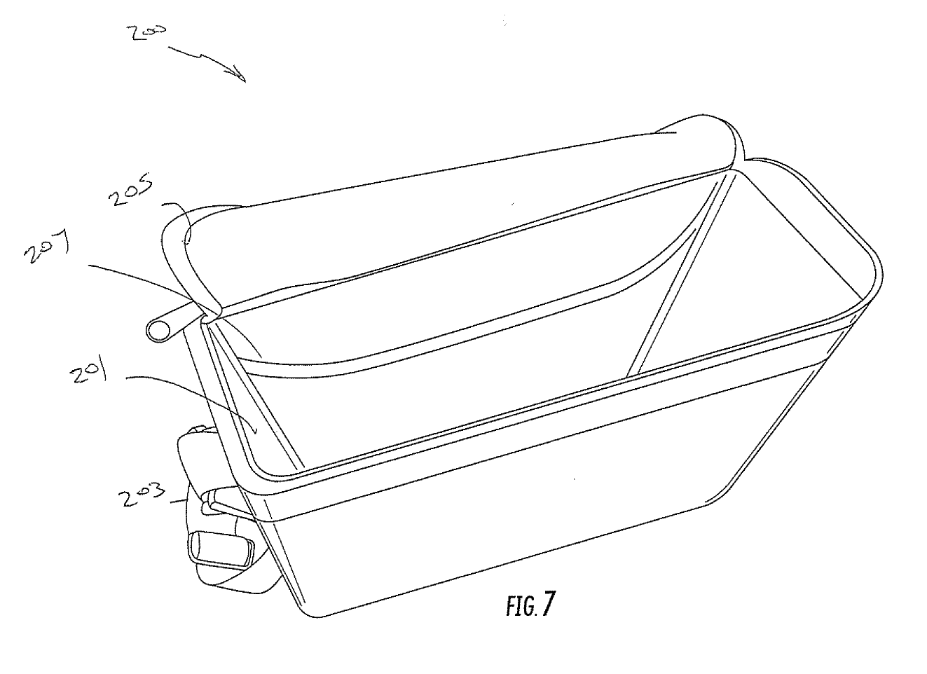

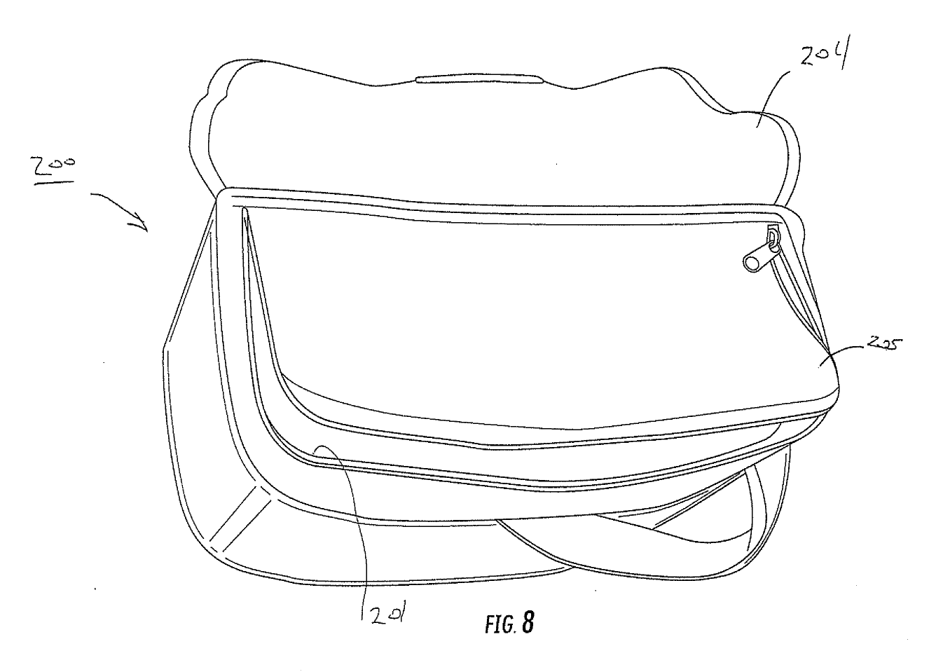

As shown in FIGS. 5-8 a storage pouch 200 or tote kit for containing system 100 is provided containing an inner opening portion region 201, a tote handle 202, a shoulder strap 203, and a main opening flap 204 having a Velcro closure and covering a zippered inner opening flap 205. There is a rear removable storage pouch 206 for storing accessories and having a Velcro closure flap. Internally, inside inner opening portion 201 is a partitioned storage region 207 for storing a preferred, external cooling pack (common gel pack), or for separately retaining thermal device 10 separate from the inner opening portion 201. In this manner, it will be recognized that storage pouch 200 similarly may serve as a kit member 120 for storing the components of the overall system 100.

In the claims, means or step-plus-function clauses are intended to cover the structures described or suggested herein as performing the recited function and not only structural equivalents but also equivalent structures. Thus, for example, although a nail, a screw, and a bolt may not be structural equivalents in that a nail relies on friction between a wooden part and a cylindrical surface, a screw's helical surface positively engages the wooden part, and a bolt's head and nut compress opposite sides of a wooden part, in the environment of fastening wooden parts, a nail, a screw, and a bolt may be readily understood by those skilled in the art as equivalent structures.

Having described at least one of the preferred embodiments of the present invention with reference to the accompanying drawings, it is to be understood that such embodiments are merely exemplary and that the invention is not limited to those precise embodiments, and that various changes, modifications, and adaptations may be effected therein by one skilled in the art without departing from the scope or spirit of the invention as defined in the appended claims. The scope of the invention, therefore, shall be defined solely by the following claims. Further, it will be apparent to those of skill in the art that numerous changes may be made in such details without departing from the spirit and the principles of the invention. It should be appreciated that the present invention is capable of being embodied in other forms without departing from its essential characteristics.

what is claimed is:

1. A discomfort management system wearable on a head of a person, comprising:
a thermal device including a plurality of compartments, each compartment containing a material that is configured to maintain a temperature that is greater than or less than an ambient temperature for a duration of time that is greater than materials forming a remainder of the device, the compartments being generally aligned in rows and columns and including fold lines therebetween such that the thermal device is configured to approximate a curvature of the head; and
a generally elongated pouch including a pocket configured to receive the thermal device therein, the generally elongated pouch configured to approximate the curvature of the head when worn and with the thermal device contained therein.

2. The discomfort management system of claim 1, further comprising a conical cap that is releasably securable to a top portion of the generally elongated pouch when worn.

3. A discomfort management system, according to claim 2, further comprising:
a storage pouch member.

4. A discomfort management kit, comprising:
one or more thermal devices including a plurality of compartments, each compartment containing a material that is configured to maintain a temperature that is greater than or less than an ambient temperature for a duration of time that is greater than materials forming a remainder of the device, the compartments being generally aligned in rows and columns and including fold lines therebetween such that the thermal device is configured to approximate a curvature of the head;
portions of the components are secured in portions spaced by a bridging material to improve flexibility;
a generally elongated pouch including a pocket configured to receive the thermal device therein, the generally elongated pouch configured to approximate the curvature of the head when worn and with the thermal device contained therein; and
a conical cap that is releasably securable to a top portion of the generally elongated pouch when worn.

5. A discomfort management kit, according to claim 4, further comprising:
a storage pouch member.
